SEQUENCE LISTING (2 sequences)

SEQ ID NO: 1 - protein, 4106 aa, Artificial
  Exemplary sequence used to illustrate invention.
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXXXXXXXXXCCXXXXXXXXXXXXXXXXXXXXXXXXXXXXXXXXXXXXXXXCXXXXXXXXXXXXXXXXXXXXXXXCXXXXXXXXXCCXXXXXXXXXXXXXXXXXXCXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 2 - protein, 4368 aa, Artificial
  Exemplary sequence used to illustrate invention.
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXXXXXXXXXXXXXXXXXXXXXXXXXXXXXXXXXXXXXXXXXXXXXXXXXXCXXXXXCXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXXXXXXXXXXXXXXXXXXXXXXXXXXXXXXXXXXXXXXXXXXXXXXXXXXCXXXXXCXXXXXXXXXXXXXXXXXXXXXXXXXXXXXXXXXXXXXXXXXXXXXXXXXXCXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

(19) United States
(12) Patent Application Publication
(10) Pub. No.: US 2013/0303631 A1
(43) Pub. Date: Nov. 14, 2013

(54) Environmentally Friendly and Aerated Topical Benefit Composition

(71) Applicants: Congling Quan, Trumbull, CT (US); Anjing Lou, Trumbull, CT (US); Badreddine Ahtchi-Ali, Trumbull, CT (US)

(72) Inventors: Congling Quan, Trumbull, CT (US); Anjing Lou, Trumbull, CT (US); Badreddine Ahtchi-Ali, Trumbull, CT (US)

(73) Assignee: CONOPCO, INC., D/B/A UNILEVER, Englewood Cliffs, NJ (US)

(21) Appl. No.: 13/469,178

(22) Filed: May 11, 2012

(51) Int. Cl. A61K 8/92; A61Q 19/08

(52) U.S. Cl. 514/773

(57) ABSTRACT

Environmentally friendly and aerated topical benefit compositions are described. The compositions are emulsified with hydrophobin and upon topical application deliver excellent sensory and absorption characteristics along with consumer desirable drying times.

### FIELD OF THE INVENTION

The present invention is directed to an environmentally friendly and aerated topical benefit composition. More particularly, the invention is directed to a composition comprising at least two immiscible liquids that are emulsified with hydrophobin as well a gas phase that is also emulsified with the hydrophobin. The composition of this invention further comprises thickening agent and skin benefit agent wherein the composition is environmentally friendly and suitable to be substantially free of surfactant and emulsifier. The composition unexpectedly displays excellent sensory benefits and rapid drying time after topical application. Moreover, such a composition is stable and surprisingly remains essentially aerated even after being stored for about three (3) months at about 45° C.

### BACKGROUND OF THE INVENTION

Topical compositions have long been used to deliver benefits to consumers. Such benefits may be pharmaceutical and/or cosmetic in nature.

Ointments, for example, have served as emollients and mainly for medicinal purposes by acting as carriers for pharmaceuticals, drugs and the like. Ointments deliver such components to the body after being topically applied.

Personal care products such as skin creams, lotions, antiperspirants and deodorants are also commonly employed to deliver benefit agents to skin of consumers by carrying sunscreens and/or other components like anti-aging, moisturizing and skin lightening ingredients.

While ointments, lotions and creams, for example, are known and widely accepted vehicles for delivering agents to skin, these vehicles can be dense, difficult to apply and/or irritants to skin. Moreover, certain vehicles may not easily dry, leaving consumers feeling wet and uncomfortable after application. Other vehicles may dry too fast, yielding tacky or cakey products of little benefit and interest to consumers.

There is an increasing interest to develop environmentally friendly compositions that are suitable to deliver skin benefit agents, provide excellent sensory benefits and that dry rapidly subsequent to being topically applied. This invention, therefore, is directed to a composition comprising at least two immiscible liquids that are emulsified with a hydrophobin. The composition further comprises thickening agent and unexpectedly provides excellent sensory benefits, hydrophobin stabilized aeration and rapid drying time after being applied. The composition of the present invention is environmentally friendly and suitable to be formulated free of surfactant and emulsifier.

### Additional Information

Efforts have been disclosed for making topical compositions. In U.S. Pat. Nos. 3,214,338 and 7,659,234 B2, medicament releasing film-forming ointments and personal care compositions with quaternary ammonium compounds are described, respectively.

Other efforts have been disclosed for making compositions with hydrophobins. In Food Hydrocolloids 23 (2009) 1877-1885 by Tchuebou-Magaia et al. (entitled, Hydrophobins Stabilized Air-Filled Emulsions for the Food Industry) and Soft Matter, 2011, 7,8248-8257 by Reger et al., (entitled, Unique Emulsions Based on Biotechnically Produced Hydrophobins), emulsions with hydrophobins are described.

Still other efforts have been disclosed for making compositions with hydrophobins. In Food Hydrocolloids 23 (2009) 366-376 by Cox et al. (entitled, Exceptional Stability of Food Foams Using Class II Hydrophobin HFBII), foam stability of aerated solutions is described.

Even other efforts have been disclosed for making compositions with hydrophobins. In U.S. Patent Application Nos. 2006/024417A, 2007/071866A, 2007/071865A and 2008/175972A, aerated compositions for human consumption are described.

U.S. Pat. No. 6,331,305 B1 describes water-in-oil cosmetic compositions with willow bark extract.

None of the additional information above describes an environmentally friendly and aerated topical benefit composition as claimed in this invention.

### SUMMARY OF THE INVENTION

In a first aspect, the present invention is directed to a composition comprising:
a) hydrophobin, the hydrophobin capable of emulsifying immiscible liquids and air within the immiscible liquids;
b) at least two immiscible liquids;
c) thickening agent; and
d) a topical benefit agent,

the composition further comprising from about 10 to about 80 percent by volume air emulsified in the composition and in the form of microbubbles having a bubble diameter from about 2 to about 150 microns.

In a second aspect, the present invention is directed to a method for making the composition of the first aspect of this invention.

In a third aspect, the present invention is directed to a method for delivering a topical benefit agent to a surface of the body by topically applying to the body the composition of the first aspect of this invention.

All other aspects of the present invention will more readily become apparent upon considering the detailed description and examples which follow.

Topical, as used herein, means external to the body. Skin, as used herein, is meant to include skin on the face, neck, chest, back, arms (including underarms), hands, legs, feet, buttocks and scalp. Hydrophobin, as used herein, means in general a natural emulsifier and cysteine-rich proteins, the same capable of emulsifying liquids like water and oil as well as air. Air, as used herein, means a gas like, for example, nitrogen, atmospheric gas, inert gas such as argon, or a gas having at least oxygen in combination with carbon dioxide, water vapor and/or nitrogen. Air is also meant to include a void created by vacuum. Thickening agent, as used herein, means an additive that thickens a composition with hydrophobin without destroying the emulsifying capabilities of the hydrophobin. An often preferred thickening agent is a polysaccharide. Topical benefit agent means an agent applied topically and suitable to be formulated in a composition with hydrophobin without destroying the emulsifying capabilities of the hydrophobin, the topical benefit agent being one that, for example, moisturizes, artificially colors, lightens and/or reduces wrinkles on skin. Such a benefit agent may also be one that, for example, provides a benefit to hair or is a pharmaceutical that acts on the skin and/or by penetrating the skin. An oil that moisturizes skin is within the scope of topical benefit agent as used herein. Thus, the oil used to make emulsion is meant to be included as a topical benefit agent. Emulsifier and surfactant are meant to mean surface-active agents that may facilitate the mixing of two or more liquids wherein the same are not meant to include the hydrophobins used in this invention. Essentially aerated means hydrophobin stabilized or emulsified air remains intact within the composition such that the density of the composition does not increase by more than 20% after being stored for about 3 months at 45° C. in a sealed container. Preferably, essentially aerated means a composition density increase from about 3 to about 15%, and most preferably, from about 5 to about 11% after storage at the aforementioned conditions.

Substantially free of means less than about 0.5%, and preferably, from about 0.001 to about 0.3% by weight in the composition and based on total weight of the composition. In an especially preferred embodiment, the composition of this invention is substantially free of emulsifier and surfactant. In a most especially preferred embodiment, no (0.0% by weight) emulsifier and surfactant will used in the composition of this invention. Environmentally friendly means being at least substantially free of emulsifier, surfactant as well as propellants such as those used in aerosol packaging.

Microbubble, as used herein, means a bubble of air present within emulsion, the bubble of air stabilized with hydrophobin. Bubble diameter means the largest measurable diameter of the microbubble. Droplet diameter means the largest measurable diameter of oil droplet emulsified with hydrophobin. The composition of this invention can be leave-on or wash-off and often is a cream, lotion, balm, deodorant, serum, foam, mousse or gel. Preferably, the composition of this invention is a leave-on composition.

Comprising, as used herein, is meant to include consisting essentially of and consisting of. For the avoidance of doubt and by way of illustration, the composition of this invention can consist essentially of emulsion, hydrophobin, topical benefit agent, air, thickening agent and minors, or the composition of this invention can consist of the same. Minors, as used herein, means typical ingredients for perfecting topical compositions such as perfecting the same for end use without having an impact on intended benefit. Minors can include, for example, preservatives, fragrances, colorants (like iron oxides), pH buffers, visual additives like speckles and/or the like. Emulsion includes a composition with water and oil, and preferably, a water-in-oil or oil-in-water emulsion. In a most preferred embodiment of this invention, the immiscible liquids are water and oil and the emulsion is an oil-in-water emulsion. All ranges identified herein are meant to include all ranges subsumed therein if, for example, reference to the same is not explicitly made.

### DETAILED DESCRIPTION OF THE PREFERRED EMBODIMENTS

The hydrophobins used herein are a well-defined class of proteins (Wessels, 1997, Adv. Microb. Physio. 38: 1-45; Wosten, 2001, Annu Rev. Microbiol. 55: 625-646) capable of self-assembly at a hydrophobic/hydrophilic interface, and having a conserved sequence:

(SEQ ID No. 1)
Xₙ-C-X₅₋₉-C-C-X₁₁₋₃₉-C-X₈₋₂₃-C-X₅₋₉-C-C-X₆₋₁₈-C-Xₘ

where X represents any amino acid, and n and m independently represent an integer. Typically, a hydrophobin has a length of up to 125 amino acids. The cysteine residues (C) in the conserved sequence are part of disulphide bridges. In the context of this invention, the term hydrophobin has a wider meaning to include functionally equivalent proteins still displaying the characteristic of self-assembly at a hydrophobic-hydrophilic interface resulting in a protein film, such as proteins comprising the sequence:

(SEQ ID No. 2)
Xₙ-C-X₁₋₅₀-C-X₀₋₅-C-X₁₋₁₀₀-C-X₁₋₅₀-C-X₀₋₅-C-X₁₋₅₀-C-Xₘ

or parts thereof still displaying the characteristic of self-assembly at a hydrophobic-hydrophilic interface resulting in a protein film. In accordance with the definition of this invention, self-assembly can be detected by adsorbing the protein to Teflon and using Circular Dichroism to establish the presence of a secondary structure (in general, α-helix) (De Vocht et al., 1998, Biophys. 3. 74: 2059-68).

The formation of a film can be established by incubating a Teflon sheet in the protein solution followed by at least three washes with water or buffer (Wosten et al., 1994, Embo. 3. 13: 5848-54). The protein film can be visualized by any suitable method, such as labeling with a fluorescent marker or by the use of fluorescent antibodies, as is well established in the art. m and n typically have values ranging from 0 to 2000, but more usually m and n in total are less than 100 or 200. The definition of hydrophobin in the context of this invention includes fusion proteins of a hydrophobin and another polypeptide as well as conjugates of hydrophobin and other molecules such as polysaccharides.

Hydrophobins identified to date are generally classed as either class I or class II. Both types have been identified in fungi as secreted proteins that self-assemble at hydrophobic-hydrophilic interfaces into amphipathic films.

Hydrophobin-like proteins have also been identified in filamentous bacteria, such as Actinomycete and Streptomyces sp. (WO01/74864; Talbot, 2003, Curr. Biol, 13: R696-R698). These bacterial proteins by contrast to fungal hydrophobins, may form only up to one disulphide bridge since they may have only two cysteine residues. Such proteins are an example of functional equivalents to hydrophobins having the consensus sequences shown in SEQ ID Nos. 1 and 2, and are within the scope of this invention.

The hydrophobins can be obtained by extraction from native sources, such as filamentous fungi, by any suitable process. For example, hydrophobins can be obtained by culturing filamentous fungi that secrete the hydrophobin into the growth medium or by extraction from fungal mycelia with 60% ethanol. It is particularly preferred to isolate hydrophobins from host organisms that naturally secrete hydrophobins. Preferred hosts are hyphomycetes (e.g. Trichoderma), basidiomycetes and ascomycetes. Particularly preferred hosts are food grade organisms, such as Cryphonectria parasitica which secretes a hydrophobin termed cryparin (MacCabe and Van Alfen, 1999, App. Environ. Microbiol 65: 5431-5435).

Alternatively, hydrophobins can be obtained by the use of recombinant technology. For example host cells, typically micro-organisms, may be modified to express hydrophobins and the hydrophobins can then be isolated and used in accordance with the present invention. Techniques for introducing nucleic acid constructs encoding hydrophobins into host cells are well known in the art. More than 34 genes coding for hydrophobins have been cloned, from over 16 fungal species (see for example WO96/41882 which gives the sequence of hydrophobins identified in Agaricus bisporus; and Wosten, 2001, Annu. Rev. Microbiol. 55: 625-646). Recombinant technology can also be used to modify hydrophobin sequences or synthesise novel hydrophobins having desired/improved properties.

Typically, an appropriate host cell or organism is transformed by a nucleic acid construct that encodes the desired hydrophobin. The nucleotide sequence coding for the polypeptide can be inserted into a suitable expression vector encoding the necessary elements for transcription and translation and in such a manner that they will be expressed under appropriate conditions (e.g. in proper orientation and correct reading frame and with appropriate targeting and expression sequences). The methods required to construct these expression vectors are well known to those skilled in the art.

A number of expression systems may be used to express the polypeptide coding sequence. These include, but are not limited to, bacteria, fungi (including yeast), insect cell systems, plant cell culture systems and plants all transformed with the appropriate expression vectors. Preferred hosts are those that are considered food grade—‘generally regarded as safe’ (GRAS).

Suitable fungal species, include yeasts such as (but not limited to) those of the genera Saccharomyces, Kluyveromyces, Pichia, Hansenula, Candida, Schizo saccharomyces and the like, and filamentous species such as (but not limited to) those of the genera Aspergillus, Trichoderma, Mucor, Neurospora, Fusarium and the like.

The sequences encoding the hydrophobins are preferably at least 80% identical at the amino acid level to a hydrophobin identified in nature, more preferably at least 95% or 100% identical. However, persons skilled in the art may make conservative substitutions or other amino acid changes that do not reduce the biological activity of the hydrophobin. For the purpose of the invention these hydrophobins possessing this high level of identity to a hydrophobin that naturally occurs are also embraced within the term “hydrophobins”.

Hydrophobins can be purified from culture media or cellular extracts by, for example, the procedure described in WO01/57076 which involves adsorbing the hydrophobin present in a hydrophobin-containing solution to surface and then contacting the surface with a surfactant, such as Tween 20, to elute the hydrophobin from the surface. See also Collen et al., 2002, Biochim Biophys Acta. 1569: 139-50; Calonje et al., 2002, Can. J. Microbiol. 48: 1030-4; Askolin et al., 2001, Appl Microbiol Biotechnol. 57: 124-30; and De Vries et al., 1999, Eur 3 Biochem. 262: 377-85.

Often, the hydrophobin is in an isolated form, typically at least partially purified, such as at least 10% pure, based on weight of solids. By “isolated form”, this means that the hydrophobin is not added as part of a naturally-occurring organism, such as a mushroom, which naturally expresses hydrophobins. Instead, the hydrophobin will typically either have been extracted from a naturally-occurring source or obtained by recombinant expression in a host organism.

Hydrophobin proteins can be divided into two classes: Class I, which are largely insoluble in water, and Class II, which are readily soluble in water.

Hydrophobins for use with the present invention are Class II hydrophobins. Preferably the hydrophobins used are Class II hydrophobins such as HFBI, HFBII, HFBIII, or Cerato ulmin.

The hydrophobin can be from a single source or a plurality of sources e.g. a mixture of two or more different hydrophobins.

The amount of hydrophobin in the total composition is preferably at least 0.001% by weight, and more preferably, at least 0.005% by weight, and most preferably, from about 0.01% by weight to about 2% by weight based on total weight of the composition and including all ranges subsumed therein. Optimally, from about 0.01 to about 1.0% by weight, and most optimally, from about 0.01 to about 0.6% by weight hydrophobin is used, based on total weight of the composition and including all ranges subsumed therein.

Regarding the immiscible liquids, the same are only limited to the extent that they are suitable for use in a composition that can be topically applied to a consumer and they are liquids at ambient temperature that can be emulsified by a hydrophobin.

Oils (liquids that are not water soluble) that are suitable for use in this invention include cyclic and linear polyorganosiloxanes as well as components generally classified as waxes, silicone liquids and silicone elastomers.

Suitable cyclic silicones include cyclic dimethylsiloxane chains containing an average of from about 3 to about 10 silicon atoms, preferably from about 4 to about 5 silicon atoms. Suitable linear silicones include the polydimethylsiloxanes containing an average of from about 3 to about 10 silicon atoms. Such silicones generally have viscosities of less than about 6 centistokes at 25° C., while the cyclic materials have viscosities of less than about 10 centistokes. Examples of silicone oils useful in the present invention include: Dow Corning 245, Dow Corning 344, Dow Corning 345, and Dow Corning 200 (fluids 5-50 cst), all made available by the Dow Corning Corporation.

Silicones classified as nonvolatile and suitable for use should have a vapor pressure over 0.1 mm Hg at 25° C., and preferably, will have an average viscosity of from about 5 to about 100,000 cps at 25° C., more preferably, from about 50 to about 10,000 cps, and most preferably, from about 400 to about 6000 cps. Lower viscosity non volatile silicone conditioning agents, however, can also be used. Viscosity can be measured by means of a glass capillary viscometer as set forth in Dow Corning Corporate Test Method CTM0004, Jul. 20, 1970.

Suitable nonvolatile silicone fluids for use herein include polyalkylsiloxanes, polyaryl siloxanes, polyalkylaryl siloxanes, polysiloxanes with amino functional substitutions, polyether siloxane copolymers, and mixtures thereof. The siloxanes useful in the present invention may be substituted and/or endcapped with any number of moieties, as long as the material remains suitable for use in a topical cosmetic product, including, for example, methyl, hydroxyl, ethylene oxide, propylene oxide, amino and carboxyl groups. Other silicone fluids having skin benefit properties may also be used. The nonvolatile polyalkyl siloxane fluids that may be used include, for example, polydimethylsiloxanes. These siloxanes are available commercially from Dow Corning as the Dow Corning 200 series. Other silicone fluids that may be used include, for example, polymethylphenylsiloxanes. These siloxanes are available, for example, from Dow Corning.

References disclosing suitable silicone fluids include U.S. Pat. No. 2,826,551, Green; U.S. Pat. No. 3,964,500, Drakoff, issued Jun. 22, 1976; U.S. Pat. No. 4,364,837, Pader; and GB-A-849,433, Woolston. In addition, Silicone Compounds distributed by Petrarch Systems Inc., 1984 provide examples.

Typically preferred silicone elastomers are organopolysiloxanes available under the INCI names of dimethicone/vinyl dimethicone cross-polymer, dimethicone crosspolymer and Polysilicone-II. Ordinarily, these materials are provided as 1-30% crosslinked silicone elastomer dissolved or suspended in a dimethicone fluid (usually, cyclomethicone). For definition purposes, crosslinked silicone elastomer means the elastomer alone rather than the total commercial compositions which include a solvent (e.g., dimethicone) carrier.

Dimethicone/vinyl dimethicone crosspolymers and dimethicone crosspolymers are available from a variety of suppliers.

Often preferred crosspolymers suitable for use include elastomers like DC 9040, 9041, 9045 and 9506, all commercially available from Dow Corning. Shin-Etsu (KSG-15, 16, 18 [dimethicone/phenyl vinyl dimethicone crosspolymer]) and Grant Industries (Gransirm line of materials) may also be used. Lauryl dimethicone/vinyl dimethicone crosspolymers from Shin-Etsu (KSG-31, 32, 41, 42, 43 and 55) can also be used.

Other oils suitable for use include natural or synthetic oils selected from mineral, vegetable, and animal oils, fats and waxes, fatty acid esters, fatty alcohols, fatty acids and mixtures thereof, which ingredients are useful for achieving emollient cosmetic properties.

Examples of oils that may be used in this invention include, for example, hydroxy-substituted C₈-C₅₀ unsaturated fatty acids and esters thereof, C₁-C₂₄ esters of C₈-C₃₀ saturated fatty acids such as isopropyl myristate, isopropyl palmitate, cetyl palmitate and octyldodecylmyristate, beeswax, saturated and unsaturated fatty alcohols such as behenyl alcohol and cetyl alcohol, hydrocarbons such as mineral oils, petrolatum and squalane, fatty sorbitan esters, lanolin and lanolin derivatives. Animal and vegetable triglycerides, can be used and they include almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grapeseed oil, shea butter, shorea butter, and sunflower seed oil and C₁-C₂₄ esters of dimer and trimer acids such as diisopropyl dimerate, diisostearylmalate, diisostearyldimerate and triisostearyltrimerate. When used, often preferred are the mineral oils, petrolatums, unsaturated fatty acids and esters thereof and mixtures thereof.

Water is typically the liquid used with the above-defined oil in order to make the desired composition (emulsion) of this invention. While tap, distilled and/or deionized water is/are generally preferred, solutions of water and water soluble ingredients may be used in emulsion formation. It is within the scope of this invention to employ water soluble ingredients, including water miscible ingredients in the water phase when making the composition of this invention. Preferred actives that are suitable for use herein are later described.

Thickening agents that may be used are limited only to the extent that they can be used in compositions suitable for applying topically to consumers and they, again, do not interfere with the emulsifier capabilities of the hydrophobin or hydrophobins used. Particularly useful are polysaccharides. Examples include starches, natural/synthetic gums and cellulosics. Representative of the starches are chemically modified starches such as sodium hydroxypropyl starch phosphate and aluminum starch octenylsuccinate. Tapioca starch is often preferred. Suitable gums include xanthan, sclerotium, pectin, karaya, arabic, agar, guar, carrageenan, alginate and combinations thereof. Suitable cellulosics include hydroxypropyl cellulose, hydroxypropyl methylcellulose, ethylcellulose and sodium carboxy methylcellulose.

Amounts of the thickener used may range from about 0.001 to about 5%, and preferably, from about 0.1 to about 3%, and most preferably, from about 0.2 to about 1.5% by weight of the composition, including all ranges subsumed therein. In a preferred embodiment, the thickening agent used is a polysaccharide. Most preferably, the thickening agent is xanthan gum.

Water-in-oil and oil-in-water emulsions are suitable for use in this invention, wherein the emulsion of this invention typically comprises from about 10% to about 85% oil, and preferably, from about 60% to about 80% by weight oil, based on total weight of the composition, including all ranges subsumed therein. Most preferably, the emulsion made with hydrophobin in this invention is a high internal phase emulsion (RIPE) with oil in the internal phase. Water will therefore make up from about 15 to about 60%, and preferably, from about 20 to about 55%, and most preferably, from about 35 to about 50% by weight of the composition, based on total weight of the composition and including all ranges subsumed therein.

When water is the continuous phase (and preferably is), hydrophobin emulsified oil has a droplet diameter within the emulsion from about 0.25 to about 20 microns, and preferably, from about 0.5 to about 15 microns, and most preferably, from about 1 to about 10 microns, including all ranges subsumed therein.

The environmentally friendly composition of this invention preferably comprises from about 10 to about 80%, and most preferably, from about 15 to about 50% by volume air, including all ranges subsumed therein. Bubble diameter for the air microbubbles emulsified within the composition of this invention is preferably from about 2 to about 150 microns, and preferably, from about 10 to about 100 microns, including all ranges subsumed therein. In an especially preferred embodiment, both oil and air are emulsified within the composition of this invention with one hydrophobin being used.

Compositions (e.g., preferred oil-in-water emulsions) of the present invention may typically include cosmetically acceptable carrier components in addition to water and the immiscible liquids used for emulsion formation. Water, nevertheless, is the most preferred carrier.

Along with the emulsion, other acceptable carrier components suitable for use in this invention may include those classified as esters. Amounts of these materials may range from about 0.1 to about 20%, and preferably, from about 0.1 to about 15%, and most preferably, from about 1 to about 10% by weight of the composition, including all ranges subsumed therein.

Among suitable esters are:
(1) Alkenyl or alkyl esters of fatty adds having 10 to 20 carbon atoms like isopropyl palmitate, isopropyl isostearate, isononyl isonanonoate, oleyl myristate, isopropyl myristate, oleyl stearate, and oleyl oleate;
(2) Ether esters such as fatty acid esters of ethoxylated fatty alcohols;
(3) Polyhydric alcohol esters such as ethylene glycol mono- and did acid esters, diethylene glycol mono- and did acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxy-ethylene sorbitan fatty acid esters;
(4) Wax esters such as beeswax, spermaceti, myristyl myristate, stearyl stearate; and
(5) Sterol esters, of which soya sterol and cholesterol fatty acid esters are examples thereof.

Preservatives can desirably be incorporated into the compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are DMDM hydantoin/iodopropynyl butyl carbamate (Glydant® Plus), iodopropynyl butyl carbamate, phenoxyethanol, methyl paraben, propyl paraben, imidazolidinyl urea, sodium dehydroacetate caprylyl glycol and benzyl alcohol. The preservatives should be selected having regard for the use of the composition and possible incompatibilities between the preservatives and other ingredients in the emulsion. Preservatives are preferably employed in amounts ranging from about 0.01% to about 2% by weight of the composition, including all ranges subsumed therein.

Conventional humectants may optionally be employed in the present invention. These are generally polyhydric alcohol-type materials. Typical polyhydric alcohols include glycerol (i.e., glycerine or glycerin), propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. Most preferred is glycerin, sorbitol, or a mixture thereof. The amount of humectant employed, when used, may range anywhere from 0.5 to 20%, preferably between 1 and 15% by weight of the composition, including all ranges subsumed therein.

Fragrances, colorants, fixatives and abrasives may optionally be included in compositions of the present invention. Each of these substances may range from about 0.05 to about 5%, preferably between 0.1 and 3% by weight.

Turning to the actives suitable for use herein, the same can include opacifiers like TiO₂ and ZnO. Such opacifiers typically have a particle size from 50 to 1200 nm, and preferably, from 50 to 350 nm. If used, such opacifiers will typically make up less than 3% by weight of the composition.

To enhance skin moisturization, actives classified as cationic ammonium compounds may optionally be used in the compositions of this invention. Such compounds include salts of hydroxypropyltri(C₁-C₃ alkyl)ammonium mono-substituted-saccaride, salts of hydroxypropyltri(C₁-C₃ alkyl)ammonium mono-substituted polyols, dihydroxypropyltri(C₁-C₃ alkyl)ammonium salts, dihydroxypropyldi(C₁-C₃ alkyl)mono(hydroxyethyl)ammonium salts, guar hydroxypropyl trimonium salts, 2,3-dihydroxypropyl tri(C₁-C₃ alkyl or hydroxalkyl)ammonium salts or mixtures thereof. In a most preferred embodiment and when desired, the cationic ammonium compound employed in this invention is the quaternary ammonium compound 1,2-dihydroxypropyltrimonium chloride. If used, such compounds typically make up from about 0.001 to about 30%, and preferably, from about 0.01 to about 15% by weight of the composition.

When cationic ammonium compounds are used, preferred additional active for use with the same are moisturizing agents such as substituted ureas like hydroxymethyl urea, hydroxyethyl urea, hydroxypropyl urea; bis(hydroxymethyl)urea; bis(hydroxyethyl)urea; bis(hydroxypropyl)urea; N,N′-dihydroxymethyl urea; N,N′-di-hydroxyethyl urea; N,N′-di-hydroxypropyl urea; N,N,N′-tri-hydroxyethyl urea; tetra(hydroxymethyl)urea; tetra(hydroxyethyl)urea; tetra(hydroxypropyl)urea; N-methyl-N′-hydroxyethyl urea; N-ethyl-N,N—N′-hydroxyethyl urea; N-hydroxypropyl-N′-hydroxyethyl urea and N,N′-dimethyl-N-hydroxyethyl urea or mixtures thereof. Where the term hydroxypropyl appears, the meaning is generic for either 3-hydroxy-n-propyl, 2-hydroxy-n-propyl, 3-hydroxy-i-propyl or 2-hydroxy-i-propyl radicals. Most preferred is hydroxyethyl urea. The latter is available as a 50% aqueous liquid from the National Starch & Chemical Division of ICI under the trademark Hydrovance.

Amounts of substituted urea, when used, in the composition of this invention range from about 0.001 to about 20%, and preferably, from about 0.01 to about 15%, and most preferably, from about 0.02 to about 10% based on total weight of the composition and including all ranges subsumed therein.

When cationic ammonium compound and substituted urea are used, in a most especially preferred embodiment at least from about 1 to about 15% glycerin external to the particle is used, based on total weight of the composition and including all ranges subsumed therein.

Compositions of the present invention may include vitamins as the desired active. Illustrative vitamins are Vitamin A (retinol) as well as retinol esters like retinol palmitate and retinol propionate, Vitamin B₂, Vitamin B₃ (niacinamide), Vitamin B₅, Vitamin C, Vitamin E, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. Total amount of vitamins when present in compositions according to the present invention may range from 0.001 to 10%, preferably from 0.01% to 1%, optimally from 0.1 to 0.5% by weight of the composition.

Octadecenedioic acid, azelaic acid, ubiquinone, dihydroxyacetone (DHA) and mixtures thereof may also be used as actives in the composition of this invention. Such compounds, when used, typically make up from about 0.2 to 4.5%, and preferably, from about 0.5 to 3% by weight of the composition, including all ranges subsumed therein.

Other optional actives suitable for use in this invention include resveratrol, resorcinols like 4-ethyl resorcinol, 4-hexyl resorcinol, 4-phenylethyl resorcinol, dimethoxytoluyl propyl resorcinol, 4-cyclopentyl resorcinol, 4-cyclohexylresorcinol, alpha- an/or beta-hydroxyacids, petroselinic acid, conjugated linoleic acid, octadecanoic acid, phenylethyl resorcinol (Symwhite 377 from Symrise), undecylenol phenylalanine (Seppi White from Seppic) mixtures thereof or the like. Such actives, when used, collectively make up from about 0.001 to about 12% by weight of the composition.

A variety of herbal extracts may optionally be included as actives in compositions of this invention. The extracts may either be water soluble or water-insoluble carried in a solvent which respectively is hydrophilic or hydrophobic. Water and ethanol are the preferred extract solvents. Illustrative extracts include those from green tea, yarrow, chamomile, licorice, aloe vera, grape seed, citrus unshui, willow bark, sage, thyme and rosemary. Soy extracts may be used and especially when it is desirable to include retinol.

Also optionally suitable for use include materials like chelators (e.g., EDTA), C₈₋₂₂ fatty acid substituted saccharides, lipoic acid, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B and Ceramide 6) as well as pseudoceramides may also be useful. Occlusives like Oilwax LC are often desired. Amounts of these materials may range from about 0.000001 to about 10%, preferably from about 0.0001 to about 1% by weight of the composition.

Sunscreen actives may also be included in compositions of the present invention. Particularly preferred are such materials as octyl salicylate, phenylbenzimidazole sulfonic acid (Ensulizole), ethylhexyl p-methoxycinnamate, available as Parsol MCX®, Avobenzene, available as Parsol 1789® and benzophenone-3, also known as Oxybenzone. Inorganic sunscreen actives may be employed such as microfine titanium dioxide, zinc oxide, polyethylene and various other polymers. Also suitable for use is octocrylene. Amounts of the sunscreen agents when present may generally range from 0.1 to 30%, preferably from 0.5 to 20%, optimally from 0.75 to 10% by weight.

Conventional buffers/pH modifiers may be used. These include commonly employed additives like sodium hydroxide, potassium hydroxide, hydrochloric acid, citric acid and citrate/citric acid buffers. In an especially preferred embodiment, the pH of the composition of this invention is from about 4 to about 8, and preferably, from about 4.25 to about 7.75, and most preferably, from about 6 to about 7.5, including all ranges subsumed therein. The composition of this invention may be a solid stick or bar. Viscosity of the composition of this invention is, however, preferably from about 1,000 to about 120,000 cps, and most preferably, from about 5,000 to 80,000 cps, taken at ambient temperature NS and a shear rate of 1 s⁻¹ with a strain controlled parallel plate rheometer made commercially available from suppliers like T.A. Instruments under the Ares name.

In an especially preferred embodiment, actives like 12-hydroxystearic acid, niacinamide, petroselinic acid, conjugated linoleic acid and/or a resorcinol is/are used.

With respect to manufacturing or preparing the environmentally friendly and aerated topical benefit composition of this invention, any mixing technique and preparation container or vessel may be used as long as the desired composition is made. For example, aeration can occur simultaneously with emulsification by injecting air into a homogenizer having water, oil and an emulsifier therein.

The inventors herein have unexpectedly discovered two-stream process that ensures excellent yield and stability of desired aerated composition. Particularly, a hydrophobin emulsified air-in-water emulsion may be made separate and distinct from an oil-in-water emulsion. Thickening agent such as a polysaccharide (e.g., xanthan gum), and preferably, a humectant (e.g., glycerin) are desired for use to prevent creaming during the emulsification process for each phase or stream. Preferably, from about 3 to about 10, and most preferably, from about 4 to about 7 times more humectant than thickening agent is used during the emulsification process. Typically, when humectant is used, from about 0.5 to about 25%, and preferably, from about 2.2 to about 15% by weight is used in the two-stream process, based on total weight of the composition and including all ranges subsumed therein.

Preferably, each emulsion made is a high internal phase emulsion with air making up from at least 75% (most preferably, 75 to 80%) by volume of the air-in-water emulsion and oil making up at least 75% (most preferably, 75 to 80%) by volume of the oil-in-water emulsion. The air-in-water emulsion may be made with a conventional apparatus or tool such as an immersion blender, continuous foaming machine and/or a high shear in line mixer. For the oil-in-water emulsion, a homogenizer and/or conventional rotor/stator mixer may be used. Final composition (i.e., aerated topical benefit composition) may be made by blending the two emulsions in a conventional mixer.

The pH of the composition of this invention is often from about 3 to about 9, and preferably, from about 5 to about 7. The viscosity of the composition of this invention is preferably from about 1,000 to about 120,000 cps, and most preferably, from about 5,000 to about 80,000 cps, including all ranges subsumed therein. Viscosity is taken with a conventional strain controlled parallel plate apparatus with temperature being ambient and shear rate being 1 s⁻¹.

When applying the composition of this invention, sensory characteristics during application are surprisingly desirable wherein the composition of this invention dries and 60 to 80% faster than conventional formulations deplete of aeration.

A wide variety of packaging can be employed to store and deliver the composition of this invention. Packaging is often dependent upon the type of personal care end-use. For instance, leave-on skin lotions and creams, shampoos, conditioners and shower gels generally employ plastic containers with an opening at a dispensing end covered by a closure. Typical closures are screw-caps, non-aerosol pumps and flip-top hinged lids. Packaging for antiperspirants, deodorants and depilatories may involve a container with a roll-on ball on a dispensing end. Alternatively these types of personal care products may be delivered in a stick composition formulation in a container with propel-repel mechanism where the stick moves on a platform towards a dispensing orifice.

The following examples are provided to facilitate an understanding of the present invention. The examples are not intended to limit the scope of the claims.

### EXAMPLE 1

The following ingredients were used to prepare an air-in-water emulsion consistent with this invention.

| Ingredient | Weight % |
|---|---|
| Hydrophobin | 0.1 |
| De-ionized water | Balance |
| Xanthan gum | 1.0 |
| Polysorbate 20 | 0.01 |
| Glycerin | 5.0 |
| Preservative | 0.12 |

The ingredients were combined within a mixing vessel and mixed in order to create a homogenous composition. Temperature of the contents varied from ambient to about 50° C. during the mixing process. A commercially available immersion blender was used to whip/mix the homogeneous composition. Mixing took place for about two (2) minutes for each 100 g of composition to create the desired air-in-water emulsion. The emulsion was about 60% air by volume with a microbubble size in the range from 2 to 50 microns.

### EXAMPLE 2

The following ingredients were used to prepare an oil-in-water emulsion consistent with this invention.

| | |
|---|---|
| Ingredient | Weight % |

Aqueous Phase

| | |
|---|---|
| Hydrophobin | 0.18 |
| De-ionized water | Balance |
| Xanthan gum | 0.1 |
| Glycerin | 0.5 |
| Preservative | 0.12 |

Oil Phase

| | |
|---|---|
| Elastomer blend (DC9041) | 35.0 |
| Cyclopentasiloxane (DC245) | 35.0 |

Emulsion (oil-in-water) was obtained after mixing the ingredients in a conventional homogenizer.

The water and oil phases were prepared separately. In this example, the aqueous phase was mixed with a high shear mixer as oil phase was gradually added thereto. A Silverson® Rotor/Stator mixer was used. Mixing was complete when the viscosity of the resulting oil-in-water emulsion had the consistency of a hand and body lotion.

### EXAMPLE 3

The emulsions prepared in Examples 1 and 2 were combined in order to produce, via a two-stream process, the environmentally friendly and aerated topical benefit composition of this invention. Conventional mixers were used to mix one (1) part by weight air-in-water emulsion to one (1) part by weight oil-in-water emulsion. A homogeneous mixture of emulsions was prepared. The resulting emulsion was 43% by volume air. The total internal phase of this aerated, oil-in-water emulsion was 64% by volume air and oil.

### EXAMPLE 4

| | | | Sample C |
|---|---|---|---|
| | Sample A | Sample B | (Control) |
| Ingredients | Weight % | | |
| Hydrophobin | 0.14 | 0.14 | 0.14 |
| De-ionized water | Balance | Balance | Balance |
| Xanthan gum | 0.55 | 0.50 | 0.50 |
| Polysorbate 20 | 0.005 | 0.01 | 0.01 |
| Glycerin | 2.75 | 2.50 | 2.50 |
| Preservative | 0.12 | 0.12 | 0.12 |
| Silicon elastomer | 18.69 | 18.69 | 18.69 |
| (DC 9041) | | | |
| Cyclopentasiloxane | 18.69 | 18.69 | 18.69 |
| (DC 245) | | | |
| Total | 100.0 | 100.0 | 100.0 |

Samples A and B were processed in order to yield environmentally friendly and aerated topical benefit compositions according to this invention. The same were prepared via two-stream processing as described herein. About 40% by volume of the resulting emulsions were air, whereby the internal phase of the same was 62% by volume was air and oil.

The control obtained was an oil-in-water emulsion deplete of aeration and prepared via conventional emulsion preparation steps. The control had an internal phase with about 37% by volume oil.

### EXAMPLE 5

Drying capabilities of Samples A-C in Example 4 were assessed. A test drying apparatus at 22° C. and relative humidity 32% was set up. Samples from A-C were applied to 2.5 cm×7.5 cm glass slides, composition film thickness of about 100 micrometers. The slides were assessed for weight loss at the times recorded where weight loss is proportionate to volatile component evaporation. Weight loss/volatile component evaporation was recorded using a commercially available microbalance.

Drying profiles are assessed in the table below. Aerated samples (A and B), made consistent with this invention, dried 60 to 80% faster than non-aerated Control C.

| Composition (Weight loss %) | | | Time to Dry |
|---|---|---|---|
| Sample A | Sample B | Sample C | (Seconds) |
| 0 | 0 | 0 | 0 |
| 7 | 7 | 3 | 100 |
| 13 | 13 | 7 | 200 |
| 20 | 20 | 12 | 300 |
| 25 | 25 | 14 | 400 |
| 29 | 29 | 18 | 500 |
| 33 | 33 | 21 | 600 |

It was also unexpectedly discovered by skilled panelists that the compositions made according to this invention not only dried faster than conventional non-aerated emulsions but also had excellent sensory characteristics consistent with the non-aerated control.

### EXAMPLE 6

The compositions of this example were made with the ingredients below. Sample D was aerated to 40% by volume air. The microbubbles possessed an average size of about 20 to 30 microns. Sample E (Control) was not aerated.

| | | Sample E |
|---|---|---|
| | Sample D | (Control) |
| Ingredients | Weight % | |
| Hydrophobin | 0.14 | 0.15 |
| De-ionized water | Balance | Balance |
| Xanthan gum | 0.55 | 0.73 |
| Preservative | 0.15 | 0.15 |
| Silicone elastomer (DC 9041) | 18.75 | 11.25 |
| Cyclopentasiloxane (DC 245) | 18.75 | 11.25 |
| Total | 100.00 | 100.00 |

Aerated and non-aerated compositions were made as described herein and tested in a sensory expert panel. Ponds® Fine Pore, a desirable Unilever commercial product containing 40 weight % elastomer gel and other skin beneficial agents, is well known for its silkiness characteristics and was used as a bench mark. All samples were applied to human subject forearms on an equal volume basis. Product absorption and silkiness were evaluated immediately after the product was applied by the panelists. About five (5) minutes after product was applied, absorption and silkiness were again evaluated.

The results from the panelists unexpectedly revealed that the compositions made according to this invention had excellent absorption and silkiness characteristics that were consistent with the Ponds® Fine Pore product and better than the control.

### EXAMPLE 7

The compositions of Samples F and G were prepared as described herein and aerated to an initial density of 0.44 g/ml and of 0.61 g/ml, respectively. Both aerated compositions were put into a 50 ml sealed graduated tube, which were stored in an oven set to 45° C. After 94 days of storage at 45° C., the density increase for both compositions was unexpectedly less than 10%. The same surprisingly shows that the aerated compositions made consistent with this invention were very stable in that they did not rapidly de-aerate.

| | Sample F | Sample G |
|---|---|---|
| Ingredients | Weight % | |
| Hydrophobin | 0.12 | 0.17 |
| De-ionized water | Balance | Balance |
| Xanthan gum | 1.00 | 0.55 |
| Glycerin | 5.00 | 2.75 |
| Preservative | 0.15 | 0.15 |
| Silicone elastomer (DC 9041) | — | 18.75 |
| Dimethicone 5 (DC 241) | — | 18.75 |
| Total | 100.00 | 100.00 |
| Initial density, g/ml, of aerated composition | 0.44 | 0.61 |
| Density, g/ml, of aerated composition after | 0.45 | 0.67 |
| being stored at 45° C. for 94 days | | |

### EXAMPLE 8

Samples H to L were prepared and aerated as described above with air at 40 volume percent. Increasing levels of xanthan gum, glycerin, starches, particles and skin benefit agents in the composition improved the thickness and sensory feel of aerated products without having a negative impact on aeration.

| | Sample | Sample | Sample | Sample | Sample |
|---|---|---|---|---|---|
| | H | I | J | K | L |
| Ingredients | Weight % | | | | |
| Hydrophobin | 0.14 | 0.14 | 0.14 | 0.14 | 0.14 |
| De-ionized water | Balance | Balance | Balance | Balance | Balance |
| Xanthan gum | 1.10 | 0.60 | 1.10 | 1.10 | 0.55 |
| Glycerin | 11.00 | 4.75 | 11.00 | 11.00 | 5.5 |
| Preservative | 0.15 | 0.15 | 0.15 | 0.15 | 0.15 |
| Hydroxypropyl | — | 0.50 | — | — | — |
| starch phosphate | | | | | |
| Tapioca starch | — | — | 2.50 | — | — |
| Polyethylene microsphere | — | — | — | 1.00 | — |
| Hydroxyethyl urea | — | — | — | — | 0.125 |
| 1,2-dihydroxypropyl- | — | — | — | — | 0.125 |
| trimonium chloride | | | | | |
| Silicon elastomer | 18.75 | 18.75 | 18.75 | 18.75 | 18.75 |
| (DC 9041) | | | | | |
| Cyclopentasiloxane | 18.75 | 18.75 | 18.75 | 18.75 | 18.75 |
| (DC 245) | | | | | |
| Total | 100.00 | 100.00 | 100.00 | 100.00 | 100 |

1. A composition consisting essentially of:
a) hydrophobin, capable of emulsifying immiscible liquids and air within the immiscible liquids;
b) two immiscible liquids one of which is water and the other is oil;
c) thickening agent selected from the group consisting of xanthan gum;
d) a topical benefit agent;
e) from about 10 to about 80 percent by volume air in the form of microbubbles having a bubble diameter from about 2 to about 100 microns: and
f) minors selected from the group consisting of preservatives, fragrances, colorants, pH buffers and visual additives
wherein no emulsifier and surfactant other than hydrophobin are used in the composition and the composition is a leave-on lotion, balm, deodorant, serum or cream.

2. The composition according to claim 1 wherein the hydrophobin makes up at least about 0.001% by weight of the composition.

3. The composition according to claim 1 wherein the composition is substantially free of propellant.

4. (canceled)

5. The composition according to claim 1 wherein the hydrophobin is a class I or class II comprising hydrophobin.

6. (canceled)

7. The composition according to claim 1 wherein the topical benefit agent is a moisturizing agent or an agent that lightens skin, moisturizes skin, or reduces wrinkles on skin.

8. The composition according to claim 4 wherein the oil comprises silicone.

9. The composition according to claim 1 wherein the hydrophobin is HFBI, HFBII, HFBIII, Cerato ulmin or a mixture thereof.

10. The composition according to claim 1 wherein the composition comprises from about 10 to about 60% by volume air.

11. The composition according to claim 1 wherein the oil makes up from about 10 to about 85% by weight of the composition in the form of droplets having a droplet diameter from about 0.25 to about 10 microns.

12. A method for treating skin comprising the step of applying to skin the composition of claim 1.

13. A method for making the composition of claim 1 comprising the steps of:
a) preparing an air-in-water emulsion with hydrophobin as the only emulsifier;
b) preparing an oil-in-water emulsion with hydrophobin as the only emulsifier; and
c) combining the air-in-water and oil-in-water emulsions the method for making the composition further comprising the step of adding to the air-in-water and water-in-oil emulsions humectant and thickening agent, said thickening agent selected from the group consisting of xanthan gum, wherein from about 3 to about 10 times more humectant than thickening agent is used in each emulsion further wherein from about 0.5 to about 25% by weight humectant is used based on total weight of the composition.

14. The composition of claim 1 wherein the composition has a density that does not increase by more than 20% after being stored for about three (3) months at 45° C. in a sealed container.

15. (canceled)

16. The composition of claim 1 wherein the composition is an oil-in-water emulsion having from about 60 to 80 percent by weight oil and the benefit agent comprises 12-hydroxystearic acid, niacinamide, conjugated linoleic acid, petroselinic acid, resorcinol, sunscreen or a mixture thereof.

17. The composition according to claim 1 wherein the benefit agent comprises caprylyl glycol, phenoxyethanol, sunscreen or a mixture thereof.
